(19) United States
(12) Patent Application Publication
(10) Pub. No.: US 2021/0007977 A1
(43) Pub. Date: Jan. 14, 2021

(54) COMPOSITIONS AND METHODS OF AMELIORATING PHARMACEUTICAL AVERSIVENESS WITH SALTS

(71) Applicant: Monell Chemical Senses Center, Philadelphia, PA (US)

(72) Inventor: Paul Breslin, Pittsburgh, PA (US)

(21) Appl. No.: 16/970,733

(22) PCT Filed: Feb. 18, 2019
(86) PCT No.: PCT/US2019/018395

§ 371 Date: Aug. 18, 2020

Related U.S. Application Data
(60) Provisional application No. 62/632,183, filed on Feb. 19, 2018.

(51) Int. Cl. A61K 9/00; A61K 31/498; A61K 31/496

(52) U.S. Cl. CPC A61K 9/0053; A61K 31/496; A61K 31/498

(57) ABSTRACT

Provided herein are compositions and methods of ameliorating pharmaceutical aversiveness via salt. In one aspect, a composition is provided comprising Praziquantel, and an effective amount of Na Gluconate which suppresses bitter taste of orally administrated Praziquantel. Additionally, a composition is provided comprising Piperaquine and an effective amount of KOH which suppresses aversiveness of orally administrated Piperaquine.

### CROSS-REFERENCE TO RELATED APPLICATIONS

This application claims the benefit of the priority of U.S. Provisional Patent Application No. 62/632,183, filed Feb. 19, 2018, which application is incorporated herein by reference.

### BACKGROUND OF THE INVENTION

Significant numbers of drugs and active pharmaceutical ingredients (APIs) on the market and in development may cause aversiveness upon oral administration, e.g., bitter taste, mouth/throat irritation, and nausea, not only to children but also to many adults. Such drugs are necessary to treat global diseases such as infections with bacteria, plasmodia, and helminths, many of which are fatal. Thus, acceptable palatability of oral medicinal products is of great importance to facilitate patient adherence with a drug regimen, particularly in underdeveloped countries where medical supervision of such adherence is lacking. Unlike adults who can swallow taste-masked tablets, children are exposed to other formulations (e.g., liquid). When in liquid form, drugs can be highly aversive and children, particularly have demonstrated little tolerance of these formulations resulting in problems with compliance. If drug compliance is not improved, millions of children will continue to die from drug-treatable diseases. This has been recognized by regulatory authorities and is becoming a key aspect of pediatric pharmaceutical development studies. See, e.g., Walsh et al, “Playing hide and seek with poorly tasting paediatric medicines: Do not forget the excipients.” Advanced Drug Delivery Reviews. 73 (2014) 14-33; and Mennella et al., “Optimizing Oral Medications for Children.” Clin Ther. 2008 Nov., 30 (11): 2120-2132.

Several approaches have been utilized to mask unpleasant tastes of APIs in pediatric oral dosage forms. However, few successes have been achieved mainly due to high complexity of taste receptors and signal pathways involved in taste sensations as well as little understanding thereof. While sweeteners and flavors are the intuitive choice, such additives, when found to be effective, harm dental health or cannot be provided to diabetic children. Similarly, when salts have been previously employed to ameliorate bitterness, the degree of bitterness suppression, if any, varied widely across bitter substances. See, e.g., P. A. S. Breslin and G. K. Beauchamp, “Suppression of Bitterness by Sodium: Variation Among Bitter Taste Stimuli.” Chemical Senses, Volume 20, Issue 6, 1 Dec. 1995, Pages 609-623, doi.org/10.1093/chemse/20.6.609. Whether or not the use of any salt can suppress bitterness of any composition does not appear to be predictable from known publications.

Modification of API solubility may improve its taste characteristics; although this is not suitable for all APIs considering their large diversity in physicochemical properties and pharmacokinetic performance. Bitter receptor antagonists or taste transduction cascade blockers offer an interesting alternative approach, the use of which, is fairly new and unproven. Resins, films, lipid barrier and other coating technology might be challenging, especially when swallowing a tablet is not an acceptable route of administration. See, e.g., Walsh et al, 2014, cited above.

Therefore, compositions and methods to effectively ameliorate pharmaceutical aversiveness continue to be needed.

### SUMMARY OF THE INVENTION

In one aspect, provided herein is a composition comprising in a suitable formulation, a drug that causes aversiveness in a subject upon oral administration and an effective amount of a selected salt which suppresses the aversiveness.

In a further aspect, provided herein is a composition comprising Praziquantel, and an effective amount of sodium (Na) gluconate which suppresses the bitter taste of orally administrated Praziquantel. In one embodiment, the effective amount is 100 mM.

In yet a further aspect, provided herein is a composition comprising Piperaquine, and an effective amount of potassium hydroxide (KOH), which suppresses aversiveness of orally administrated Piperaquine. In one embodiment, the aversiveness is selected from the group consisting of bitterness, sourness, astringency and nausea. In one embodiment, the effective amount is 1M.

In one aspect, a method is provided herein to improve drug regimen compliance and/or suppress aversiveness of drug orally administrated to a subject. In one embodiment, the method comprises administering the drug and an effective amount of salt orally in the subject.

In still another aspect, methods are provided for reducing the aversive qualities of a drug by formulating the API of the drug with an effective amount of a salt which suppresses the aversiveness.

Other aspects and embodiments will be readily apparent based on the information described herein.

### BRIEF DESCRIPTION OF THE DRAWINGS

FIGS. 1A to 1K provide perceived bitterness intensities of racemic praziquantel (diamonds) or L-praziquantel (squares) dissolved in di water at concentrations illustrated on the x axis (n=11) as described in Examples 1 and 2. Each figure represents data collected from one subject.

FIGS. 2A to 2G provide perceived bitterness intensities of racemic praziquantel at concentrations illustrated on the x axis and either dissolved in di water (diamonds) or admixed with 100 mM Na Gluconate (squares) (n=7) as described in Examples 1 and 3. Each figure represents data collected from one subject.

FIGS. 3A to 3G provide perceived bitterness intensities of L-praziquantel at concentrations illustrated on the x axis and either dissolved in di water (diamonds) or admixed with 100 mM Na Gluconate (Squares) (n=7) as described in Examples 1 and 3. Each figure represents data collected from one subject.

FIGS. 4A to 4J provide perceived intensities of sensation (bitterness, sourness, astringency, and nausea) of piperaquine (diamonds) or neutralized piperaquine (squares) at concentration illustrated on the x axis (n=10) as described in Examples 1 and 4. Each figure represents data collected from one subject.

### DETAILED DESCRIPTION OF THE INVENTION

Provided herein are compositions and methods of ameliorating pharmaceutical aversiveness thus facilitating patient adherence to a drug regimen. The aversiveness is ameliorated when certain salts are added to selected bitter-tasting or nausea-inducing pharmaceutical compounds (e.g., drugs or APIs, as discussed and defined below).

### 1. Definitions and Components of the Methods and Compositions

Unless defined otherwise in this specification, technical and scientific terms used herein have the same meaning as commonly understood by one of ordinary skill in the art and by reference to published texts, which provide one skilled in the art with a general guide to many of the terms used in the present application.

The terms “amelioration”, “reduction”, “decrease”, “suppression”, or any grammatical variation thereof as used herein may refer to at least about 5%, at least about 10%, at least about 20%, at least about 30%, at least about 40%, at least about 50%, at least about 60%, at least about 70%, at least about 80%, at least about 90%, or at least about 95% of subjects tested showing an intensity of indicated sensation lower than the reference given. In certain embodiment, the terms “amelioration”, “reduction”, “decrease”, “suppression”, or any grammatical variation thereof as used herein may refer to an intensity of indicated sensation which is less than about 95%, less than about 90%, less than about 80%, less than about 70%, less than about 60%, less than about 50%, less than about 40%, less than about 30%, less than about 20%, less than about 10%, or less than about 5% of the reference given.

As used herein, the term “subject” as used herein means a mammalian animal, including a human, a veterinary or farm animal, a domestic animal or pet. In one embodiment, the subject of these methods and compositions is a human. In a further embodiment, the subject of these methods and compositions is a child. As used herein, “child” or “children” refers to a human whose age is 0 month to 18 years, including a baby who is 0 to 12 months old; a toddler who is 1 to 3 years old; a preschool child who is 3 to 5 years old; a grade-schooler who is 5 to 12 years old; and a teen who is 12 to 18 years old. In certain embodiments, the subject of these methods and composition is an adult. In yet a further embodiment, the subject of these methods and compositions is a senior adult who is beyond 65 years old. Still other suitable subjects include, without limitation, non-murine, rat, canine, feline, porcine, bovine, ovine, non-human primate and others.

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be included and described using “consisting of” or “consisting essentially of” language.

As used herein, the term “about” or “˜” means a variability of 10% from the reference given, unless otherwise specified.

As used herein, the term “aversiveness” or any grammatical variation thereof refers to an unpleasant sensation selected from the group consisting of bitter taste (bitterness), sourness, astringency and nausea. As used herein, the term “sensation” refers to a perception associated with stimulation of a drug in gastrointestinal tract (e.g., mouth or tongue), including without limitation, taste, astringency and nausea. Conventional methods of quantifying an aversiveness are known to one of skill in the art. See, e.g., Breslin et al, “Suppression of Bitterness by Sodium: Variation Among Bitter Taste Stimuli.” Chemical Senses, Volume 20, Issue 6, 1 Dec. 1995, Pages 609-623, doi.org/10.1093/chemse/20.6.609; Peyrot des Gachons, C. et al, (2011). “Bitter taste induces nausea.” Current Biology, 21, R247-248 PMID 21481757; Keast, R. S. J. and P. A. S. Breslin. (2005) “Bitterness suppression with zinc sulfate and Na-cyclamate: a model of combined peripheral and central neural approaches to flavor modification”. Pharmaceutical Science, 22, 1970-1977. PMID: 16132352; Breslin P A, and Tharp C D. “Reduction of saltiness and bitterness after a chlorhexidine rinse”, Chem Senses. 2001 February; 26(2):105-16; and Keast, R. S. J. and P. A. S. Breslin. (2002) “Modifying the bitterness of selected oral pharmaceuticals with cation and anion series of salts.” Pharmaceutical Research, 19, 1019-1026. PMID: 12180534, each of which is incorporated herein in its entirety. Methods of quantifying an aversiveness are also provided herein in the Examples.

As used herein, the term “administration” or any grammatical variations thereof refers to delivery of composition described herein to a subject. “Oral administration” or any grammatical variations thereof refers to an administration where composition described herein is taken by the subject through mouth. In one embodiment, oral administration may include without limitation, enteral administration, wherein the composition is taken through mouth and absorbed in the gastrointestinal tract; buccal administration wherein the composition is dissolved inside the cheek; sublabial administration, wherein the composition is dissolved under the lip; and sublingual administration, wherein the composition is dissolved under the tongue. Furthermore, the form of administration can be liquid (i.e., solutions or suspensions), soluble or dispersible tablets, oral wafers, chewable tablets, or orodispersible tablets.

As used herein, “an effective amount” refers to a concentration of a salt in the formulation of a drug, a concentration of a salt in mouth, or a molar ratio of a drug to salt, which suppresses aversiveness of the orally administrated drug compared to the oral administration of the drug only. In one embodiment, “an effective amount” might also refer to an amount of a salt when mixed with a drug and administrated orally, suppresses aversiveness of the drug. In a further embodiment, the salt is mixed with a drug in a liquid formulation. In one embodiment, the effective amount is about 1 mM to about 10 mM, about 10 mM to about 50 mM, about 50 mM to about 100 mM, about 100 mM to about 250 mM, about 250 mM to about 500 mM, about 500 mM to about 750 mM, about 750 mM to about 1 M, about 1 M to about 2 M, about 2 M to about 5 M, about 5 M to about 10 M. In another embodiment, the effective amount is about 100 mM. In yet another embodiment, the effective amount is about 1 M. In certain embodiment, the effective amount is about 1 mg to about 2 mg, about 2 mg to about 5 mg, about 5 mg to about 10 mg, about 10 mg to about 50 mg, about 50 mg to about 100 mg, about 100 mg to about 250 mg, about 250 mg to about 500 mg, about 500 mg to about 1 g, about 1 g to about 2 g, about 2 g to about 5 g, about 5 g to about 10 g.

In certain embodiments, the effective amount is defined as the molar ratio of API (or Drug) to salt, which is about 0.00003 (3×10⁻⁵) to about 0.5 (5×10⁻¹), or any number including and between these numbers. In one embodiment, the effective amount is a molar ratio of the API/drug to the salt of about 0.0001 (1×10⁻⁴) to about 0.0010 (1×10⁻³) or any number including and between these numbers. In another embodiment, the effective amount is a molar ratio of the API/drug to the salt of about 0.001 (1×10⁻³) to about 0.010 (1×10⁻²) or any number including and between these numbers. In yet another embodiment, the effective amount is a molar ratio of the API/drug to the salt of about 0.01 to about 0.10 or any number including and between these numbers. In a further embodiment, the effective amount is a molar ratio of the API/drug to the salt of about 0.1 to about 0.5 or any number including and between these numbers.

As used herein, the term “salt” refers to a chemical compound consisting of one, two or more positively charged cation(s) and one, two or more negatively charged anion(s). In some embodiments, the cation is selected from the group consisting of H⁺, Na⁺, K⁺, Ca²⁺, Cs⁺, and Zn²⁺. Sodium (Na⁺) was shown to suppress perceived bitterness of urea, quinine HCl, caffeine, MgSO₄, Amiloride, and KCl, presumably by acting at the peripheral taste level and not by cognitive effects. However, the degree of bitterness suppression by sodium varied widely across bitter substances and cannot be predicted by one of skill in the art, and thus needs to be investigated by a case-by-case basis. See, e.g., Mennella et al, 2008, and Breslin et al, 1995, as cited herein, each of which is incorporated herein in its entirety. In some embodiments, the anion is selected from the group consisting of Cl⁻, Gluconate, Glutamate, Adenosine Monophosphate, Phosphatidate, Diphosphates, Phosphate, Citrate, Malate, Tartarate, Ascorbate, and Hydroxide. In one embodiment, salt is Na Gluconate. In another embodiment, salt is KOH. In another embodiment, salt is a base other than KOH, including but not limited to, NaOH and Na₂CO₃.

As used herein, the terms “drug” “pharmaceutical drug” and “pharmaceuticals” are used interchangeably and refer to a composition comprising a chemical or biological compound which has a physiological effect when administrated in a subject in need (e.g., the active pharmaceutical ingredient or API), and a pharmaceutical acceptable carrier. In certain embodiments, the term “drug” refers to an API. In other embodiments, in which aversion is caused by a component of the pharmaceutical composition other than the API, the term “drug” encompasses any ingredient of the composition, including but not limited to the API. As used herein, a physiological effect refers to stopping or reversing progression of a disease (e.g., infection with bacteria, plasmodia, and helminths). The physiological effect might include but not limited to clearing a parasite, a bacterium, a fungal, or a virus, from the subject.

As used herein, the term “API” is short for active pharmaceutical ingredient and refers to a chemical or biological compound which has a physiological effect when administrated in a subject in need. In one embodiment, the API is selected from the group consisting of anti-malarial, anti-protozoal, anti-parasitic, anti-viral, anti-retroviral, anti-bacterial, and anti-fungal, anti-cold and flu symptoms, anti-algesia, and anti-allergy drugs. In one embodiment, the API is in its levorotatory form. In another embodiment, the API is in its dextrorotatory form. In yet another embodiment, the API is a racemic mixture of levorotatory form and dextrorotatory form.

In one embodiment, the API is a major pharmaceutical compound for treating algesia; worms, viral or bacterial infections; or, cold, flu or allergy symptoms. In one embodiment, the API is selected from the group consisting of Praziquantel, Piperaquine, Dihydroartemisinin, Ritonavir, Tenofovir, Acetaminophen, Diphenhydramine, Nicotine, Caffeine, Dextromethorphan, Guaifenesin and Loratidine. In one embodiment, the API is an Over-The-Counter (OTC) pharmaceutical or a drug sharing both structural and functional similarities thereto.

In one embodiment, the API is Praziquantel or a drug sharing both structural and functional similarities thereto. In a further embodiment, the API is racemic mixture of praziquantel. In yet a further embodiment, the API is L-praziquantel. In another embodiment, the API is D-praziquantel.

In one embodiment, the API is Piperaquine or a drug sharing both structural and functional similarities thereto. In another embodiment, the API is Dihydroartemisinin or a drug sharing both structural and functional similarities thereto. In yet another embodiment, the API is Ritonavir or a drug sharing both structural and functional similarities thereto. In one embodiment, the API is Tenofovir or a drug sharing both structural and functional similarities thereto.

In one embodiment, the API is caffeine or a drug sharing both structural and functional similarities thereto. In one embodiment, the API is nicotine or a drug sharing both structural and functional similarities thereto. In one embodiment, the API is caffeine, nicotine, while the drug is a nicotine gum. In one embodiment, caffeine may be used to enhance analgesia and alertness and nicotine comes in nicotine gums to aid in smoking cessation.

As used herein, structural and functional similarities refer to two or more APIs which share at least about 80% identity of chemical groups and are able to achieve same physiological effect with a variability of less than about 10% when administered to a subject in need. Conventional methods of analyzing structure and functions of an API are known to one of skill in the art, including but not limited to mass spectrometry (MS), electron microscopy, and various pharmacokinetics and physiological analysis.

In another embodiment, the API is Acetaminophen (i.e., Paracetamol) or a drug sharing both structural and functional similarities thereto. In another embodiment, the API is Diphenhydramine or a drug sharing both structural and functional similarities thereto. In yet another embodiment, the API is Dextromethorphan or a drug sharing both structural and functional similarities thereto. In a further embodiment, the API is Guaifenesin (i.e., guaiphenesin or glyceryl guaiacolate) or a drug sharing both structural and functional similarities thereto. In one embodiment, the API is Loratidine or a drug sharing both structural and functional similarities thereto.

Other suitable APIs might be selected. In one embodiment, the API is an anti-algesia or analgesic compound, for example Paracetamol (acetaminophen) or Nonsteroidal anti-inflammatory drug (NSAIDs). Other suitable anti-algesia compound can be found at e.g., www.drugs.com/drug-class/analgesics.html and en.wikipedia.org/wiki/Analgesic.

In one embodiment, the API is an anti-allergy compound.

In one embodiment, the API is for alleviating the symptoms of cold or flu in a subject in need, comprising reduction of the frequency of cough by at least about 25%, at least about 50%, at least about 75%, or at least about 90%; relief of the muscle pain, chills, dehydration, fatigue, fever, flushing, loss of appetite, body ache, sweating, congestion, runny nose, or sneezing, by at least about 25%, at least about 50%, at least about 75%, or at least about 90%.

In one embodiment, the API is an anti-parasitic compound, for example, Antiprotozoals, Antihelminthic, Antinematodes, Anticestodes, Antitrematodes, Antiamoebics, and Antifungals. Other suitable anti-parasitic compound can be found at e.g., F. Matthew Kuhlmann, James M. Fleckenstein, 157—Antiparasitic Agents, Infectious Diseases (Fourth Edition), 2017, Pages 1345-1372.e2, Volume 2, Available online 12 Aug. 2016; en.wikipedia.org/wiki/Antiparasitic; and www.cyto.purdue.edu/cdroms/cyto2/17/chmrx/anthelmi.htm.

In one embodiment, the API may be an antiviral for example, Abacavir, Acyclovir (Aciclovir). Other suitable anti-parasitic compound can be found at e.g., www.fda.gov/Drugs/DrugSafety/InformationbyDrugClass/ucm100228.htm#ApprovedDrugs, en.wikipedia.org/wiki/List_of_antiviral_drugs, or www.emedexpert.com/lists/antivirals.shtml, or www.drugs.com/drug-class/antiviral-agents.html, www.cdc.gov/flu/professionals/antivirals/links.htm, and www.fda.gov/drugs/drugsafety/informationbydrugclass/ucm100228.htm.

In one embodiment, the API is an antibacterial, for example, Vancomycin, Aminoglycosides, Aminoglycosides, Ansamycins, Carbacephem, Cephalosporins, Glycopeptides, Lincosamides, Lipopeptide, Macrolides, Monobactams, Oxazolidinones, Penicillins, Penicillin combinations, Quinolones/Fluoroquinolones, Sulfonamides, Tetracyclines, Drugs against mycobacteria, and others. Other suitable antibacterial can be found in a variety of publicly available publications, such as websites, e.g., www.emedicinehealth.com/antibiotics/article_em.htm, or www.drugs.com/article/antibiotics.html, en.wikipedia.org/wiki/List_of_antibiotics, or www.merckmanuals.com/professional/infectious-diseases/bacteria-and-antibacterial-drugs/overview-of-antibacterial-drugs, or www.emedexpert.com/lists/antibiotics.shtml, or www.emedicinehealth.com/antibiotics/article_em.htm#7_types_of_antibiotics, among others.

In one embodiment, the API is an antifungal medication, including but not limited to, Polyene antifungals, Hamycin, Imidazoles, Triazoles, Thiazoles, Allylamines, Echinocandins. Other suitable antifungal medications can be found in publicly available sources, e.g., www.nhs.uk/conditions/antifungal-medicines/, www.drugs.com/drug-class/antifungals.html, https://www.drugs.com/drug-class/topical-antifungals.html, www.livestrong.com/article/27116-list-antifungals/, www.emedexpert.com/lists/antifungals.shtml, www.merckmanuals.com/professional/infectious-diseases/fungi/antifungal-drugs, en.wikipedia.org/wiki/Antifungal, and/or masshealthdruglist.ehs.state.ma.us/MHDL/pubtheradetail.do?id=28.

In one embodiment, the API is an antimalarial medication, including without limitation, e.g., quinine, Chloroquine and chloroquine phosphate, Amodiaquine and its combination with artesunate or sulfadoxine-pyrimethamine; Pyrimethamine and its combination with sulfadoxine. Other suitable antimalarial medication can be found in publicly available sources such as www.mayoclinic.org/diseases-conditions/malaria/diagnosis-treatment/drc-20351190, www.drugs.com/condition/malaria.html, www.drugs.com/drug-class/antimalarial-combinations.html, en.wikipedia.org/wiki/Antimalarial_medication, www.cdc.gov/malaria/travelers/drugs.html, www.nap.edu/read/11017/chapter/11, www.medindia.net/drugs/medical-condition/malaria.htm, www.canada.ca/en/public-health/services/travel-health/drugs-generic-trade-name-treatment-prevention-malaria.html, and/or emedicine.medscape.com/article/221134-medication.

### 2. Compositions

In one embodiment, provided herein is a composition comprising a drug which causes aversiveness in a subject upon oral administration, and an effective amount of a salt consisting of a cation and an anion, wherein the composition suppresses aversiveness of the drug. In certain embodiments, the drug is in a liquid formulation. In other embodiments, the salt is added during preparation of the drug, which may be in a solid or semi-solid or other formulation. Wherever herein the specification refers to a liquid formulation, it is understood that this formulation is an example only and that other formulations are also encompassed. In certain specific embodiments shown in the examples below, these drugs include but are not limited to praziquantel and piperaquine. Similarly, the salts include, without limitation, any combination of the cations (e.g., Na⁺, K+, and Zn²⁺), and the anions (e.g., Gluconate, Glutamate, adenosine monophosphate, and hydroxide). The aversiveness that is ameliorated comprises perceived bitterness, sourness, astringency, and nausea. See, e.g., Examples 2-4.

In one embodiment, the API is Praziquantel or a drug sharing both structural and functional similarities thereto, and the salt is Na Gluconate. In a further embodiment, the effective amount of Na Gluconate is 100 mM in a liquid formulation comprising praziquantel as the API. In yet a further embodiment, the concentration of Praziquantel or a drug sharing both structural and functional similarities thereto is about 0.001 mg/mL to about 0.152 mg/mL. In one embodiment, the concentration of Praziquantel or a drug sharing both structural and functional similarities thereto is about 0.0019 mg/mL, about 0.0056 mg/mL, about 0.0169 mg/mL, about 0.0507 mg/mL, or about 0.152 mg/mL. In some embodiments, Praziquantel or a drug sharing both structural and functional similarities thereto is in its levorotatory form. In some embodiments, Praziquantel or a drug sharing both structural and functional similarities thereto is a racemic mixture.

In certain embodiments, the API is Piperaquine or a drug sharing both structural and functional similarities thereto, and the salt is KOH. In a further embodiment, the effective amount of KOH is 1M in a liquid formulation comprising piperaquine as the API. In yet a further embodiment, the concentration of Piperaquine or a drug sharing both structural and functional similarities thereto is about 4 mg/mL to about 32 mg/mL. In some embodiment, the concentration of Piperaquine or a drug sharing both structural and functional similarities thereto is about 4 mg/mL, about 8 mg/mL, about 16 mg/mL, about 26 mg/mL, or about 32 mg/mL. In certain embodiments, the aversiveness is selected from the group consisting of bitterness, sourness, astringency and nausea. Conventional methods of measuring and quantifying aversiveness is known to one of skill in the art, e.g., Keast, R. S. J. and P. A. S. Breslin. (2002) “Modifying the bitterness of selected oral pharmaceuticals with cation and anion series of salts.” Pharmaceutical Research, 19, 1019-1026. PMID: 12180534

In one embodiment, provided herein is a composition comprising about 0.001 mg/mL to about 0.152 mg/mL Praziquantel and 100 mM Na Gluconate in a liquid formulation, wherein the composition suppresses bitter taste of Praziquantel.

In another embodiment, provided herein is a composition comprising about 4 mg/mL to about 32 mg/mL Piperaquine and 1M KOH in a liquid formulation, wherein the composition suppresses aversiveness of Piperaquine. In certain embodiments, the aversiveness is selected from the group consisting of bitterness, sourness, astringency and nausea.

In certain embodiments, a suitable pharmaceutical acceptable carrier may be readily selected by one of skill in the art in view of the API used, and may include without limitation, a diluent, an excipient, a vector, a stabilizer, a buffer, a preservative, a sweetener, a flavor, a taste receptor antagonist, a taste transduction cascade blocker, and/or an adjuvant. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

It should be understood that any embodiment of the composition provided herein may be utilized in any other embodiment, composition, method or kit described herein.

### 3. Methods and Kits

In one aspect, a method is provided herein to suppress aversiveness upon oral administration of a drug in a subject. In one embodiment, the method comprises administering the drug and an effective amount of salt orally to the subject. In one embodiment, the salt is in a liquid formulation of the drug. In one embodiment, the salt is added to the formulation of the drug upon administration. In another embodiment, the salt is administered to the subject prior to the administration of the drug.

In one embodiment, the method comprises preparing and administering the composition as described herein, and in the Examples below.

In one embodiment, a kit is provided herein comprising an effective amount of a salt as described herein to mix with a drug as described herein to suppress aversiveness of the drug upon oral administration.

It should be understood that any embodiment of the method or the kit provided herein may be utilized in any other embodiment, composition, method or kit described herein.

### EXAMPLES

The following examples are provided to illustrate certain aspects of the claimed invention. The invention is not limited to these examples. These examples demonstrate the levo-praziquantel (L-PZQ) was found less bitter than the racemic mixture of levo-praziquantel (L-PZQ) and dextro-pratiquantel (D-PZQ). Furthermore, adding potassium hydroxide (KOH) to piperaquine reduced bitterness, sourness, astringency and nausea.

### Example 1

### Methods and Materials

Methods of evaluating the sensory properties in the Examples 2 to 4 were adopted from Breslin P A, and Tharp C D, “Reduction of saltiness and bitterness after a chlorhexidine rinse,” Chem Senses. 2001 February; 26(2):105-16. More details are illustrated below.

Subjects participated in the study after providing informed consent. Gender of the subjects was balanced between male and female. The subjects were adults with ages ranging from 20 years old to 50 years old. The only exclusion criteria, since it was a rinse and spit study, was the ability to follow instructions, demonstrate proper use of the labeled magnitude scale, and to taste. The participants were asked to refrain from eating, drinking, or chewing gum for 1 hr prior to testing. Subjects were trained to use the General Labeled Magnitude Scale (gLMS) following standard published procedures. See. e.g., Breslin P A, and Tharp C D. “Reduction of saltiness and bitterness after a chlorhexidine rinse,” Chem Senses. 2001 February; 26(2):105-16; Keast and Breslin, “Bitterness suppression with zinc sulfate and Na-cyclamate: a model of combined peripheral and central neural approaches to flavor modification.” Pharm Res. 2005 November ; 22(11):1970-7. Epub 2005 Aug. 26; Green et al, “Evaluating the ‘Labeled Magnitude Scale’ for measuring sensations of taste and smell.” Chem Senses. 1996 June; 21(3):323-34; Barry G. Green, et al, “Derivation and evaluation of a semantic scale of oral sensation magnitude with apparent ratio properties,” Chemical Senses, Volume 18, Issue 6, 1 Dec. 1993, Pages 683-702; and Bartoshuk L M, “Comparing sensory experiences across individuals: recent psychophysical advances illuminate genetic variation in taste perception.” Chem Senses. 2000 August; 25(4):447-60.

Racemic praziquantel and L-praziquantel were diluted/dissolved in deionized (di) filtered water upon use into various concentrations, including 0.0019 mg/mL, 0.0056 mg/mL, 0.0169 mg/mL, 0.0507 mg/mL, and 0.152 mg/mL. Racemic praziquantel and L-praziquantel of the above indicated concentrations were also prepared in 100 mM Na Gluconate. Piperaquine was prepared and dissolved in deionized filtered water or in 1M KOH to a final concentration of 4 mg/mL, 8 mg/mL, 16 mg/mL, 26 mg/mL, or 32 mg/mL, respectively. All solutions were stored on ice or at 4° C. to 8° C. and brought up to room temperature prior to testing.

The following testing protocol was performed. The tested solutions were provided to the subjects in random order. Subjects rinsed with di water at least four times over a 2-minute period prior to tasting each solution. The subjects were instructed to wear nose clips to eliminate olfactory input, pour the whole solution in their mouth for 3-5 seconds, and rate the perceived sensation (including bitterness, sourness, astringency and nausea) intensity of the solution while it remained in the mouth, prior to expectorating. Taste intensity was recorded on a computerized gLMS, analyzed and plotted.

### Example 2

### Sensory Profiles of Target Medications and Validation of Taste Blocking Compounds—Levo-Praziquantel vs. Racemic Praziquantel

The sensory properties of racemic praziquantel and levo-praziquantel (1-praziquantel, L-PZQ) were assessed over a broad range of concentrations.

Eleven subjects participated in this study. Racemic praziquantel and L-praziquantel in di water of 0.0019 mg/mL, 0.0056 mg/mL, 0.0169 mg/mL, 0.0507 mg/mL, and 0.152 mg/mL were prepared and provided to the subject in random order according to the protocols described in Example 1.

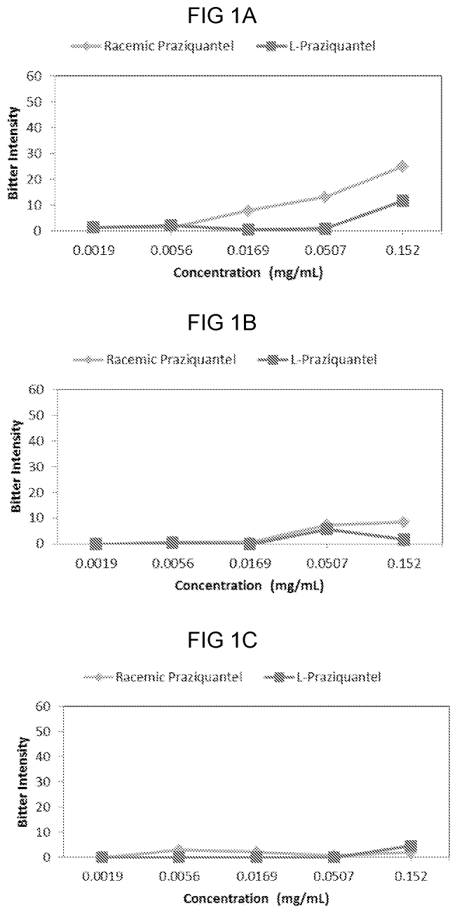

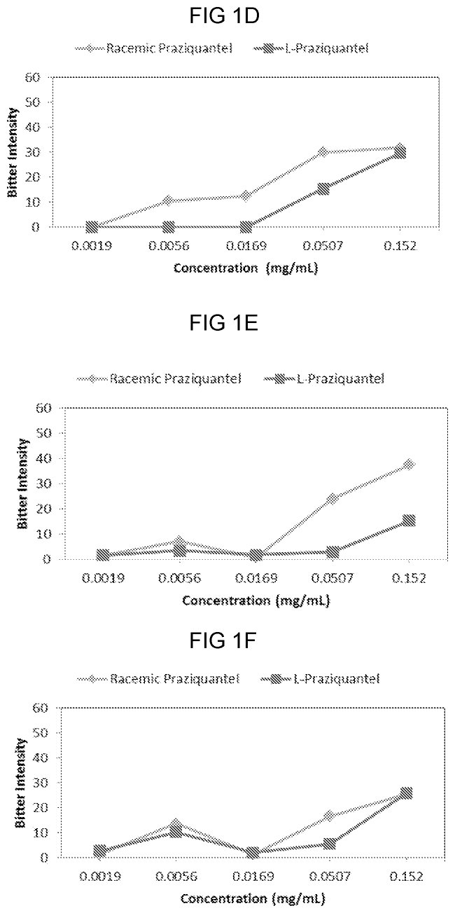

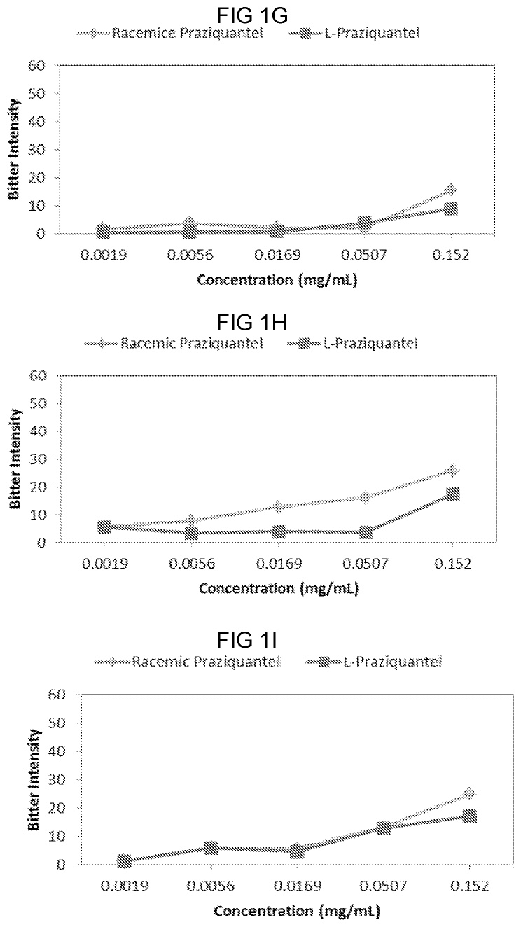

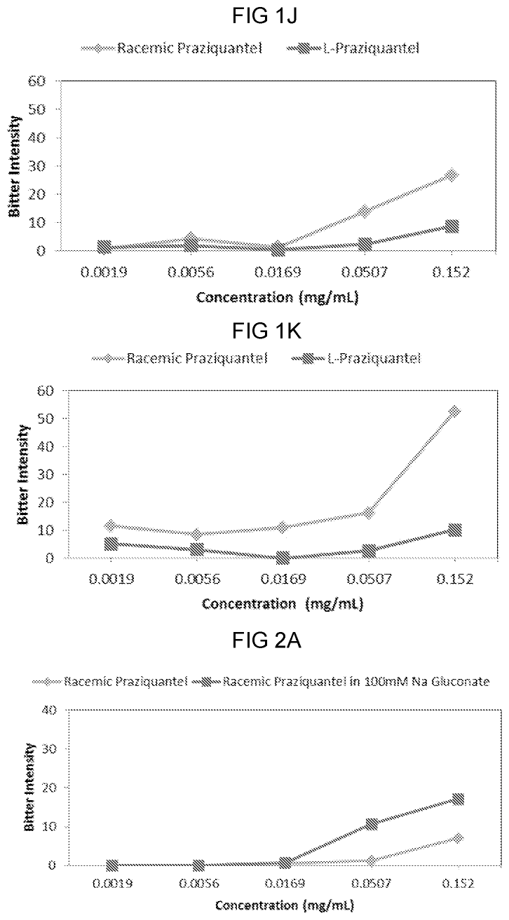

Results were recorded and plotted in FIGS. 1A to 1H, each figure of which represents data collected from one subject. Briefly, 10 out of 11 subjects, except the subject shown in FIG. 1C, reported a reduced bitter intensity of L-praziquantel compared to that of racemic praziquantel at certain concentrations. Especially, at a concentration of 0.0507 mg/mL, 1-praziquantel was scored as less bitter compared to the racemic mixture by 7 out of 11 subjects; while at a concentration of 0.152 mg/mL, L-praziquantel was less bitter as reported by 8 out or 11 subjects.

These results demonstrate that although individual differences exist in the perception of bitterness, L-praziquantel is more palatable compared to the racemic mixture due to a reduced bitterness perception.

### Example 3

### Sensory Profiles of Target Medications and Validation of Taste Blocking Compounds—Praziquantel vs. Praziquantel in 100 mM Na Gluconate

Amelioration on perceived bitterness of racemic or levorotatory praziquantel using sodium gluconate was assessed.

Seven subjects participated this study. 0.0019 mg/mL, 0.0056 mg/mL, 0.0169 mg/mL, 0.0507 mg/mL, and 0.152 mg/mL of racemic praziquantel in di water or in 100 mM Na Gluconate was prepared and provided to the subjects in random order according to the protocols described in Example 1.

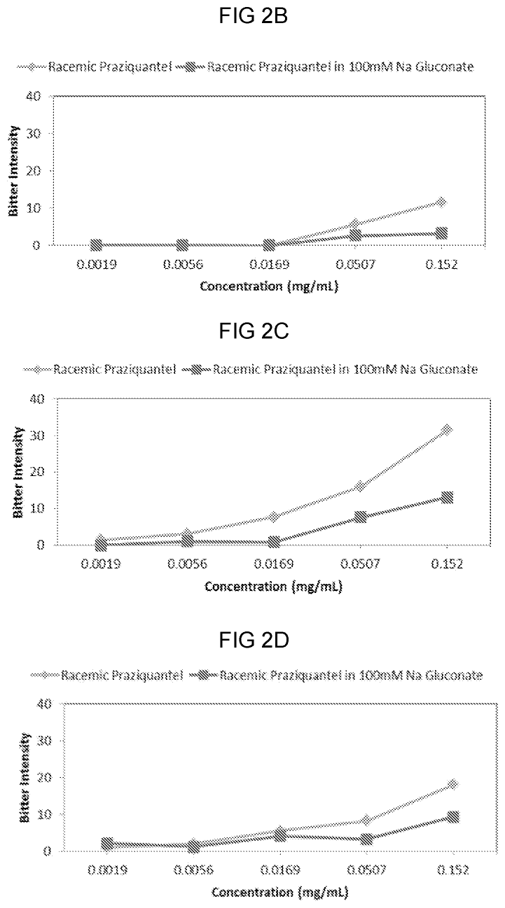

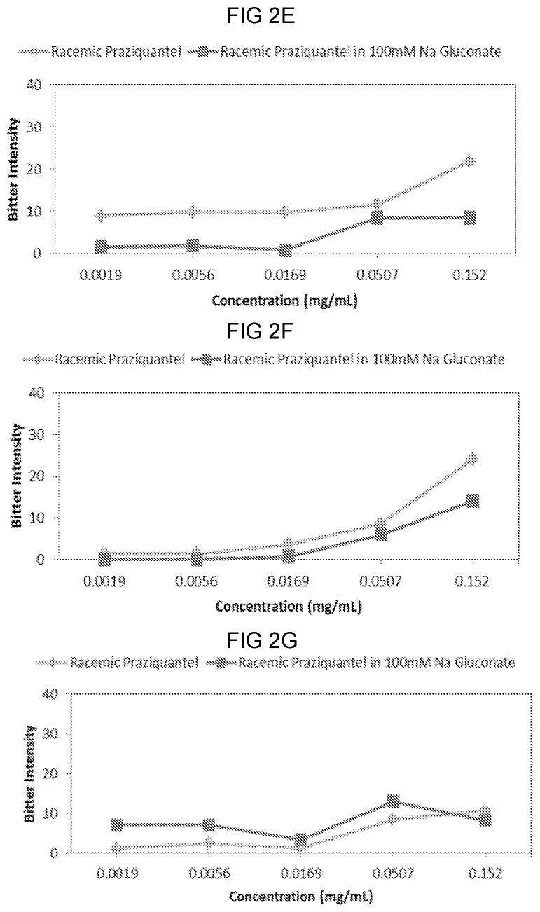

The recorded bitter intensities were plotted in FIGS. 2A to 2G, each figure of which represents data collected from one subject. Briefly, 5 out of 7 subjects perceived Na Gluconate reduced bitterness of racemic praziquantel compared to that of racemic praziquantel alone at two or more tested concentrations of praziquantel. At a concentration of 0.0507 mg/mL, racemic praziquantel was scored as less bitter in Na Gluconate compared to that in di water by 5 out of 7 subjects; while at concentration of 0.152 mg/mL, racemic praziquantel in Na Gluconate was less bitter as reported by 6 out of 7 subjects.

Similarly, 7 subjects were given 0.0019 mg/mL, 0.0056 mg/mL, 0.0169 mg/mL, 0.0507 mg/mL, and 0.152 mg/mL of L-praziquantel in di water or in 100 mM Na Gluconate according to the protocols described in Example 1.

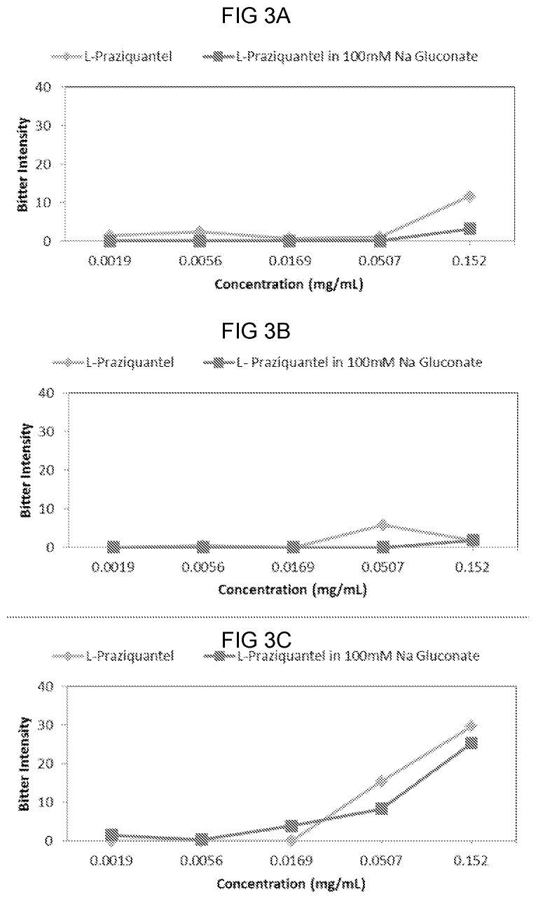

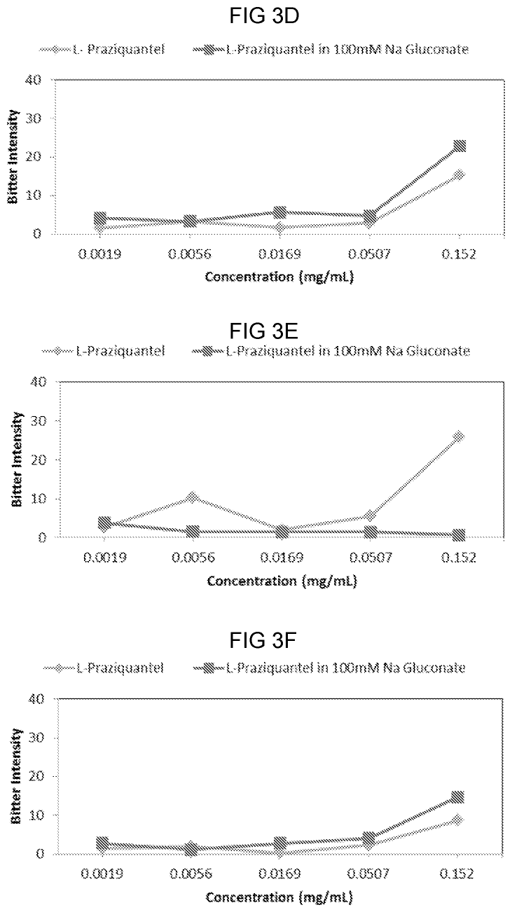

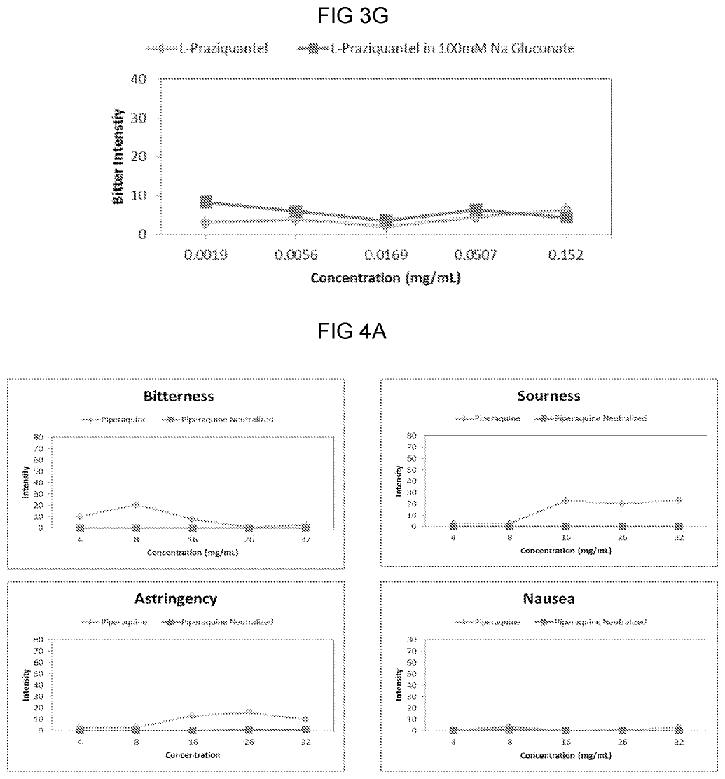

The recorded bitter intensities of each subject were plotted in FIGS. 3A to 3G individually. Four (4) out of 7 subjects showed that Na Gluconate reduced bitterness of L-praziquantel compared to that of racemic praziquantel alone at certain praziquantel concentrations. Three (3) out of 7 subjects were observed to report reduced bitterness of 0.0507 mg/mL l-praziquantel in 100 mM Na Gluconate compared to that in di water; while a reduced bitter intensity was reported by 4 out of 7 subjects when given 0.152 mg/mL l-praziquantel in Na Gluconate compared to that in di water.

Taking the results shown above together, 100 mM Na Gluconate can successfully decrease perceived bitterness of praziquantel (both racemic and levorotatory) in most subjects. These results indicate that Na Gluconate can serve as a bitter blocker for praziquantel, increase palatability of praziquantel, and therefore facilitate patient adherence. Various concentrations of Na Gluconate, ranging from approximately 25 mM to 100 mM, are under investigation for their effects on perceived sensations, including bitterness, sourness, stringency and nausea, of both racemic and levorotatory praziquantel. The same experimental settings as described in these examples with the cation replaced by Zn²⁺, Ca²⁺ and/or the anion replaced by Cl⁻, monophosphate, glutamate, and sulfate are performed to evaluate their ability of ameliorating aversiveness caused by the drug(s).

### Example 4

### Sensory Profiles of Target Medications and Validation of Taste Blocking Compounds—Piperaquine vs. Neutralized Piperaquine

Influence of pH neutralization on piperaquine sourness and astringency, as well as bitterness and nausea were examined.

Ten (10) subjects were given piperaquine in di water or neutralized with 1M KOH, at final concentrations of 4 mg/mL, 8 mg/mL, 16 mg/mL, 26 mg/mL, and 32 mg/mL and assessed according to the protocols described in Example 1.

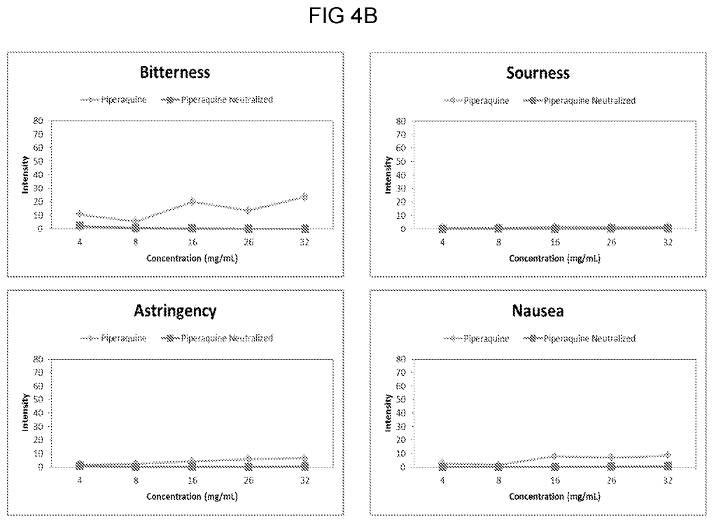

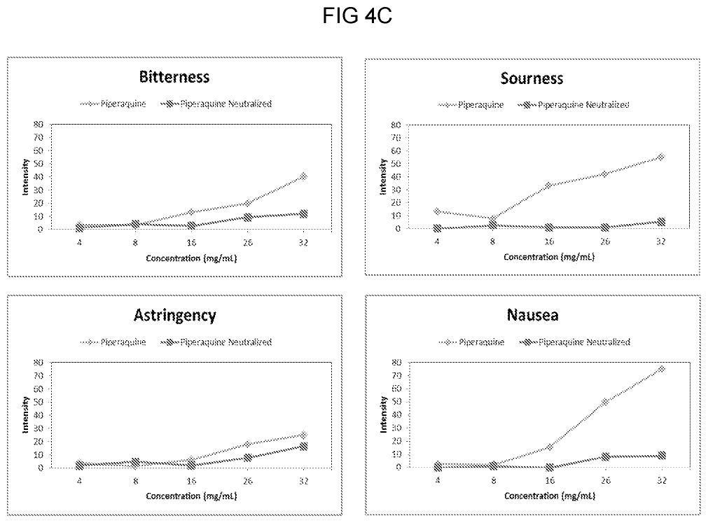

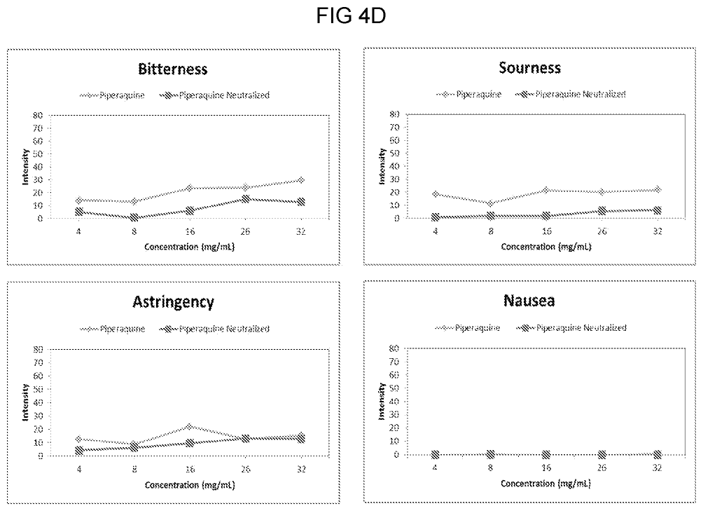

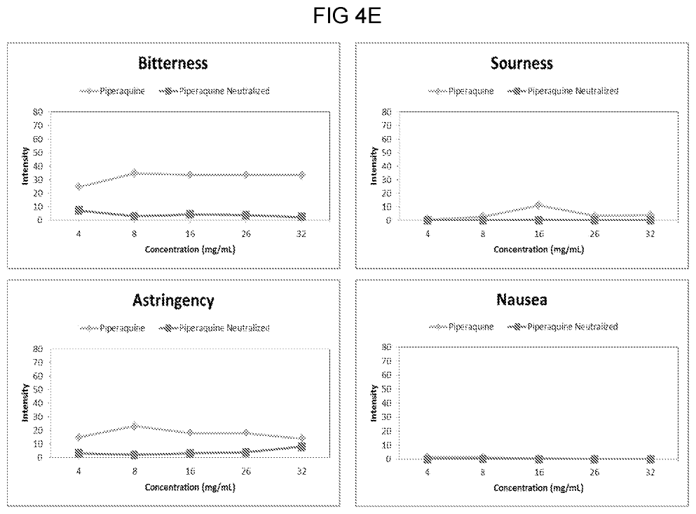

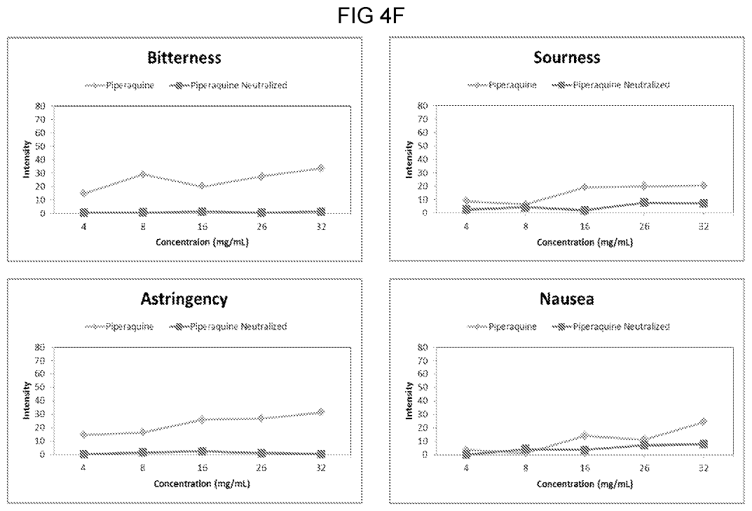

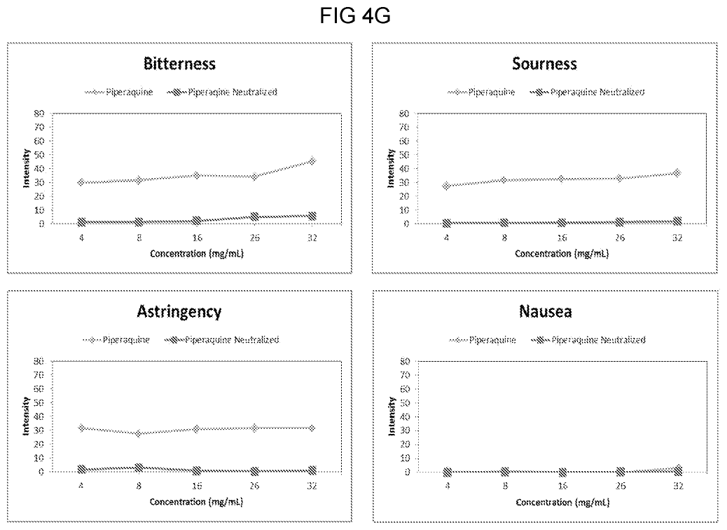

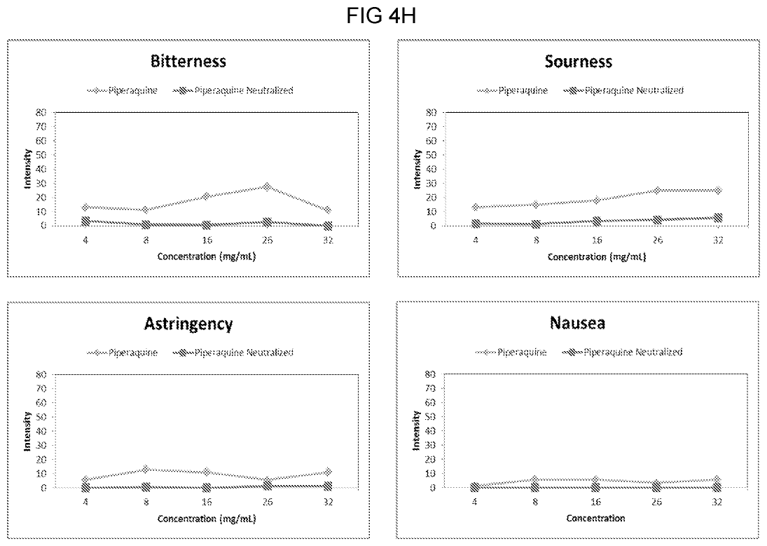

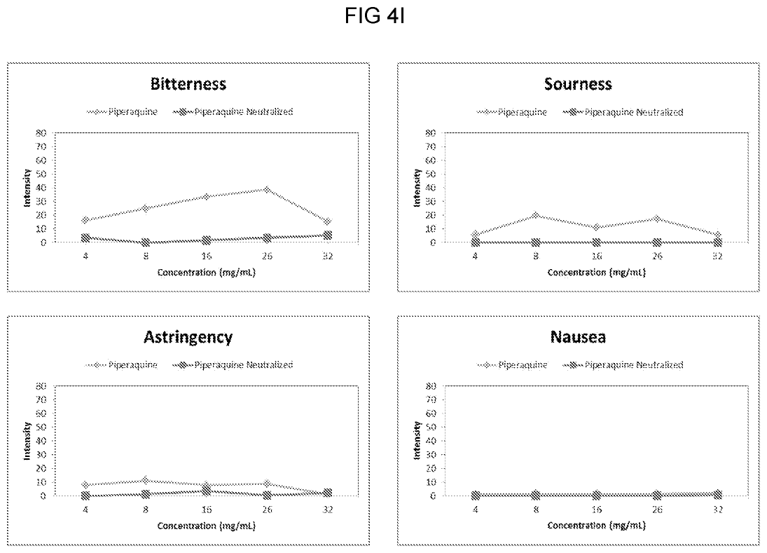

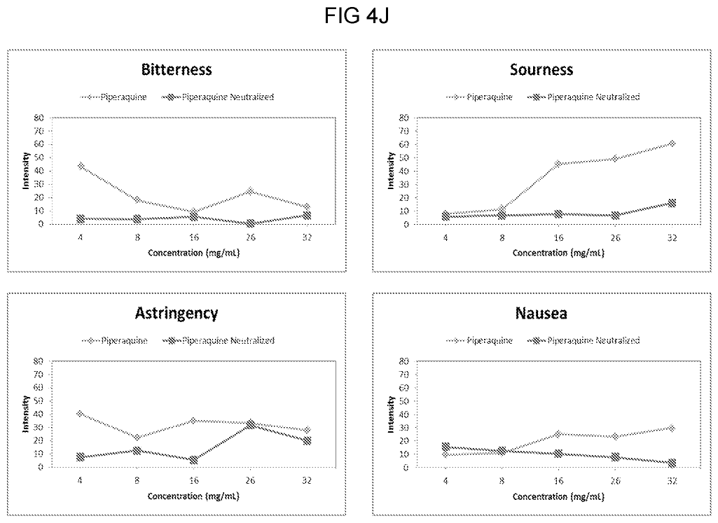

The recorded sensation intensities were plotted in FIGS. 4A to 4J, each figure of which represents data collected from one subject. Individual differences in the perception of bitterness, sourness, astringency and nausea were observed. pH neutralization of piperaquine by adding potassium hydroxide (KOH) reduced bitterness (10/10 subjects), sourness (9/10 subjects), astringency (10/10 subjects) and nausea (5/10 subjects).

All publications cited in this specification, as well as U.S. Provisional Patent Application No. 62/632,183, are incorporated herein by reference. While the invention has been described with reference to particular embodiments, it will be appreciated that modifications can be made without departing from the spirit of the invention. Such modifications are intended to fall within the scope of the appended claims.

1. A composition comprising a drug which causes aversiveness in a subject upon oral administration, and a salt consisting of a cation and an anion, wherein the composition suppresses aversiveness of the drug, and wherein molar ratio of the drug to the salt is 0.00003 to 0.5

2. The composition according to claim 1, wherein the drug is selected from the group consisting of anti-malarial, anti-protozoal, anti-parasitic, anti-viral, anti-retroviral, anti-bacterial, anti-fungal, anti-cold and flu symptoms, anti-analgesia, and anti-allergy drugs.

3. The composition according to claim 1, wherein the drug is Praziquantel or a drug sharing both structural and functional similarities thereto.

4. The composition according to claim 1, wherein the drug is Piperaquine or a drug sharing both structural and functional similarities thereto.

5. The composition according to claim 1, wherein the drug is Dihydroartemisinin or a drug sharing both structural and functional similarities thereto.

6. The composition according to claim 1, wherein the drug is Ritonavir or a drug sharing both structural and functional similarities thereto.

7. The composition according to claim 1, wherein the drug is Tenofovir or a drug sharing both structural and functional similarities thereto.

8. The composition according to claim 1, wherein the drug in its levorotatory form.

9. The composition according to claim 1, wherein the cation is selected from the group consisting of H⁺, Na⁺, K⁺, Ca²⁺, Cs⁺ and Zn²⁺.

10. The composition according to claim 1, wherein the anion is selected from the group consisting of Cl⁻, Gluconate, Glutamate, Adenosine Monophosphate, Phosphatidate, Diphosphates, Phosphate, Citrate, Malate, Tartarate, Ascorbate, and Hydroxide.

11. The composition according to claim 1, wherein the drug is Praziquantel or a drug sharing both structural and functional similarities thereto, and wherein the salt is Na Gluconate.

12. The composition according to claim 11, wherein the concentration of Na Gluconate is 100 mM.

13. The composition according to claim 11, wherein the concentration of Praziquantel or a drug sharing both structural and functional similarities thereto is about 0.001 mg/mL to about 0.152 mg/mL.

14. (canceled)

15. The composition according to claim 4, wherein the drug is Piperaquine or a drug sharing both structural and functional similarities thereto, and wherein the salt is KOH.

16. The composition according to claim 15, wherein the concentration of KOH is 1M.

17. The composition according to claim 15, wherein the concentration of Piperaquine or a drug sharing both structural and functional similarities thereto is about 4 mg/mL to about 32 mg/mL.

18. The composition according to claim 1, wherein the aversiveness is selected from the group consisting of bitterness, sourness, astringency and nausea.

19. The composition according to claim 1, wherein the drug and salt are in a liquid formulation.

20. A composition comprising about 0.001 mg/mL to about 0.152 mg/mL Praziquantel and 100 mM Na Gluconate in a liquid formulation, wherein the composition suppresses bitter taste of Praziquantel.

21. A composition comprising about 4 mg/mL to about 32 mg/mL Piperaquine and 1M KOH in a liquid formulation, wherein the composition suppresses aversiveness of Piperaquine.

22. (canceled)
